(19) United States
(12) Patent Application Publication
(10) Pub. No.: US 2009/0306685 A1
(43) Pub. Date: Dec. 10, 2009

(54) NONINVASIVE TRANS-CATHETER METHOD AND APPARATUS FOR REMOTE SUTURE PLACEMENT SUCH AS FOR SEPTAL DEFECT REPAIR, LEFT ATRIAL APPENDAGE CLOSURE, PACEMAKER ELECTRODE PLACEMENT, MITRAL VALVE REPAIR, AND OTHER INNER-CARDIAC AND INNER-ARTERIAL APPLICATIONS

(75) Inventor: Brian J. Fill, Tarentum, PA (US)

Correspondence Address: BLYNN L. SHIDELER;THE BLK LAW GROUP, 3500 BROKKTREE ROAD, SUITE 200, WEXFORD, PA, 15090, US

(73) Assignee: Ension, Inc., Pittsburgh, PA (US)

(21) Appl. No.: 12/136,757

(22) Filed: Jun. 10, 2008

(51) Int. Cl. A61B 17/12

(52) U.S. Cl. 606/148

(57) ABSTRACT

A noninvasive trans-catheter method and apparatus for remote suture placement can be used in a variety of inner cardiac applications. For example, a method of non-invasive transcatheter atrial septal defect repair comprises the steps of: advancing a positioning member along a catheter into the Atrial Septal Defect, wherein at least one suture deploying lumen is coupled to the positioning member with a piercing member within the suture deploying lumen; deploying the positioning member within the Atrial Septal Defect to align each suture deploying lumen with tissue adjacent the Atrial Septal Defect; and piercing the tissue adjacent the Atrial Septal Defect with the piercing member to secure a suture line through the tissue. A repair patch may be advanced along suture lines to repair the defect and secured into place with the suture lines. The apparatus is also applicable for left atrial appendage closure, mitral valve repair and pacemaker electrode placement.

The present invention claims priority of U.S. Provisional Patent Application Serial No. [REDACTED] entitled “Noninvasive Trans-Catheter Method and Apparatus for Remote Suture Placement such as for Septal Defect Repair, Left Atrial Appendage Closure, Pacemaker Placement, Mitral Valve Repair, and other Inner-Cardiac and Inner-Arterial Applications” filed [REDACTED].

### BACKGROUND OF THE INVENTION

1. Field of the Invention

The present invention relates to noninvasive, trans-catheter method and apparatus for remote suture placement, such as non-invasive inner-cardiac suture placement for Atrial Septal Defect (ASD) repair with a suture secured patch.

2. Background Information

Atrial Septal Defect (ASD)

The septum is a wall that separates the heart's left and right sides. Septal defects are sometimes called a “hole” in the heart. A defect between the heart's two upper chambers (the atria) is called an atrial septal defect (ASD). When there is a large defect between the atria, a large amount of oxygen-rich (red) blood leaks from the heart's left side back to the right side. Then this blood is pumped back to the lungs, despite already having been refreshed with oxygen. This is inefficient, because already-oxygenated blood displaces blood that needs oxygen. Many people with this defect have few, if any, symptoms.

Closing an ASD in childhood can prevent serious problems later in life. The long-term outlook is excellent. If atrial septal defects are diagnosed in adulthood, the defect is also repaired. Rarely, if the defect is left un-repaired if there's pulmonary hypertension (high blood pressure in the lungs).

In order to understand ASD, it first helps to review some basics about the way a healthy heart typically works. The heart has four chambers: The two lower pumping chambers are called the ventricles, and the two upper filling chambers are the atria. In a healthy heart, blood that returns from the body to the right-sided filling chamber (right atrium) is low in oxygen. This blood passes to the right-sided pumping chamber (right ventricle), and then to the lungs to receive oxygen. The blood that has been enriched with oxygen returns to the left atrium, and then to the left ventricle. It's then pumped out to the body through the aorta, a large blood vessel that carries the blood to the smaller blood vessels in the body. The right and left filling chambers are separated by a thin shared wall, called the atrial septum.

There are three classified types of atrial septal defects: secundum, primum, and sinus venosus. Secundum atrial septal defect is by far the most common, representing 80% of all ASD's. It is caused by the failure of a part of the atrial septum to close completely during the development of the heart. This results in an opening in the wall between the atria (a “hole” between the chambers). Primum atrial septal defects are part of the spectrum of the AV canals, and are frequently associated with a split in the leaflet of the valve, or so called cleft mitral valve. The sinus venosus atrial septal defect occurs at the junction of the superior vena cava and the right atrium. This represents the floor of the right atrium where blood returns from the upper extremities and head to enter the right atrium. These may frequently be associated with anomalous drainage of the pulmonary veins. This means that one or more of the pulmonary veins, which normally carry oxygenated blood from the lungs back to the left atrium, enters the right atrium instead. There are numerous types of abnormal pulmonary venous connections.

Twenty percent of atrial septal defects will close spontaneously in the first year of life. One percent of atrial septal defects become symptomatic in the first year, with an associated 0.1% mortality. There is a 25% lifetime risk of mortality in un-repaired atrial septal defects. The risk factors associated with increased mortality include the development of a condition in which the pulmonary arteries become thickened and obstructed due to increased flow, from left to right for many years (pulmonary vascular obstructive disease). This is why we electively close ASD's which have not closed spontaneously by school-age.

Certain types of ASD's (sinus venosus and primum varieties) have no chance of spontaneous closure, and patients with these types of ASD's are not candidates for transcatheter closure because of the location of the ASD. Currently, open heart surgery is indicated for patients with these types of ASD's.

ASDs occur during fetal development of the heart and are present at birth. During the first weeks after conception, the heart develops. If a problem occurs during this process, a hole in the atrial septum may result. In some cases, the tendency to develop an ASD may be genetic. There can be genetic syndromes that cause extra or missing pieces of chromosomes that can be associated with ASD. For the vast majority of children with a defect, however, there's no clear cause of the ASD.

The size of an ASD and its location in the heart will determine what kinds of symptoms a child experiences. Most children who have ASDs seem healthy and appear to have no symptoms. Generally, children with an ASD feel well and grow and gain weight normally. Infants and children with larger, more severe ASDs, however, may possibly show some of the following signs or symptoms: poor appetite, poor growth, fatigue, shortness of breath, lung problems and infections, such as pneumonia. If an ASD is not treated, health problems can develop later, including an abnormal heart rhythm (known as an atrial arrhythmia) and problems in how well the heart pumps blood. As children with ASDs get older, they may also be at an increased risk for stroke, since a blood clot that develops can pass through the hole in the wall between the atria and travel to the brain. Pulmonary hypertension (high blood pressure in the lungs) may also develop over time in older patients with larger untreated ASDs. Fortunately, most children with ASD are diagnosed and treated long before the heart defect causes physical symptoms. Because of the complications that ASDs can cause later in life, pediatric cardiologists often recommend closing ASDs early in childhood.

Once an ASD is diagnosed, treatment will depend on the child's age and the size, location, and severity of the defect. In children with very small ASDs, the defect may close on its own. Larger ASDs usually won't close, and must be treated medically or surgically.

Surgical Repair of ASD

Indications for surgical repair of an atrial septal defect are right ventricular overload (due to flow from the left atrium into the right atrium), a shunt fraction greater than 2.0 as estimated by echocardiography (the amount of blood going to the pulmonary circulation divided by the amount of blood going out to the systemic circulation), and elective closure prior to a child starting school. The surgical treatment options for an ASD closure include direct suture repair, which is reserved for small atrial septal defects, and the more common patch repair. The material utilized for patch closure of ASD's may be the patient's own pericardium, commercially available bovine pericardium, or synthetic material (Gore-Tex, Dacron).

The surgical approach to the atrial septal defect is somewhat dependent upon its location. In general, three surgical approaches may be undertaken: median sternotomy (midline sternal-splitting incision); right thoracotomy (going between the ribs on the right side); and submammary (under the breast tissue on the right front of the chest). All types of ASD's may be approached adequately through a median sternotomy or right thoracotomy. The submammary incision may be the most cosmetic, but makes some ASDs difficult to repair. The primary benefits of the submammary and thoracotomy incisions are cosmetic in nature.

The term “minimally invasive surgery” for repair of atrial septal defects usually refers to repair of the defect using the same techniques as open heart surgical repair (that is, using the heart-lung machine or “cardiopulmonary bypass”), but performing the operation through a much smaller incision. Most children can successfully undergo this type of repair through a small (3-4 inches) incision in the sternum (breastbone). In general, the postoperative course in the hospital is shorter (2-3 days), due to less incisional pain and discomfort.

Once the pericardium is opened, regardless of the choice of incisions, the patient is placed on cardiopulmonary bypass (using the heart-lung machine) and blood is diverted away from the right atrium. Cardioplegia (a mixture of medications and nutrients) with high potassium is then administered after the aorta is clamped, thus stopping the heart. The right atrium is then opened to allow access to the atrial septum below.

The breastbone is then separated to expose the heart. The patient is then placed on the heart-lung bypass machine, a device that provides blood flow to the body and “bypasses” the patient's heart and lungs. Diverting the heart's blood flow to the bypass pump allows the surgeon to open the heart, drain it and operate on the internal structures. The heart-lung bypass machine provides continuous oxygenated blood to the other organ systems during the open-heart surgery. Once the patient is on bypass, the actual surgical repair begins. Dependent upon the size and location of the defect, it may be closed directly with sutures or with a patch. In the latter case, a patch may be created by the surgeon from the patient's own pericardial tissue or a synthetic material such as Goretex® may be used. The patch is then sutured into place to close the defect.

The atrial incision is then closed with sutures. The aortic cross clamp is removed, and after normal ventilation is resumed, the patient is warmed and a stable rhythm is achieved, the patient may be weaned from cardiopulmonary bypass. A single drainage tube is placed and the chest is closed.

The results of surgical repair is a mortality of less than one percent (actually risk of surgical morbidity (5%) and mortality (<1%)), and average hospital stay is four days. Optimal timing for surgery in the asymptomatic child remains prior to starting grade school. The asymptomatic child with an atrial septal defect deserves close follow-up by the pediatrician and pediatric cardiologist, with constant involvement of the cardiovascular surgeon. Should a patient become symptomatic with failure to thrive, or persistent complaints (malaise, respiratory infections, etc.), early surgical intervention would be warranted.

Trans-Catheter Management of ASD

In light of this history, interventional cardiologists explored the possibility of transcatheter closure of the atrial septal defect. This technique involves implantation of one of several occlusion devices (basically self-expanding wire frames with integrated fabric material) using heart catheterization methods in the cardiac catheterization laboratory, without the need for cardiopulmonary bypass (heart-lung machine), and without the need to stop the heart. Defects amenable to such device therapy tend to be smaller (less than 20 to 25 mm [¾ to 1 inch] diameter). Importantly, for many prior art ASD patch designs these lesions must be centrally located within the atrial septum. Defects at the very upper or lower edges of the atrial septum (called ostium primum or sinus venosus) are not good candidates for this procedure.

The usual procedure is very similar to standard heart catheterization. Briefly, flexible long tubes (or catheters) are inserted into the veins and arteries in the groin or neck. We use the knowledge that in all human beings, these vessels are directly attached to the heart, and this is the standard access technique used in all patients. Routine pressures and oxygen levels in all of the chambers of the heart are then obtained. Angiograms (pictures taken following dye injection) are performed to determine the size of the chambers, the size of the defect, and its location within the heart. Using a balloon catheter of a known diameter, the defect is then sized in comparison to the balloon, so that the device appropriate for that particular patient can be chosen. The device is then advanced into the heart through an introducer sheath (larger, less flexible tube).

With most of the presently used devices, the introducer sheath distal tip is situated very close to the defect position where half of the device is allowed to expand on one side of the atrial septum, and the second half of the device is allowed to expand on the opposite side, forming a sort of “sandwich” of the defect. The device generally eliminates or lessens the shunting flow through the defect. Integrated fabric of the device stimulates normal tissue growth in and over the defect, and within six to eight weeks any residual shunting is eliminated. This is how, for example, these devices can be used in growing children; though the device itself does not grow, the tissue that covers the device does, and will continue to grow as the child grows. The entire procedure is performed under general anesthesia, and the actual implantation of the device is performed using transesophageal echocardiographic guidance (ultrasound pictures using a probe introduced into the esophagus for improved imaging of the heart structures) and/or fluoroscopic imaging. The major advantage of this technology is its relative non-invasive approach. Patients are usually hospitalized overnight, and many return to work or school within 1-2 days.

For a representation of a device see http://www.amplatzer.com/ describing the “Amplatzer septal occluder” as a self-expandable, double disc device made from a Nitinol wire mesh. The two discs are linked together by a short connecting waist corresponding to the size of the ASD. In order to increase its closing ability, the discs and the waist are filled with polyester fabric. The polyester fabric is securely sewn to each disc by a polyester thread. ASD closure devices are also sold under the CardioSeal, and the Angel Wings brands.

Compiling data for all the presently tested devices, the complication rate following transcatheter ASD occlusion is approximately 5%. These complications include the routine risks of cardiac catheterization such as vascular injury (damage to the veins and arteries of the leg), particularly in cases where larger device introducer systems need to be used. Sometimes, problems with blood clotting or excessive bleeding may be seen, particularly in younger patients. A complication unique to this technology may be the possibility of clot formation on the device itself, with the risk of breakage of the clot causing stroke, or a clot into the vessels of the lung (pulmonary embolus). At present, these problems are addressed by using adequate doses of aspirin or warfarin following the procedure, and by using heparin during the procedure to reduce the clotting factors within the blood. The aspirin or warfarin is used for three to six months.

The length of and need for antibiotic prophylaxis against infections in the heart (bacterial endocarditis) vary amongst investigators and devices lasting from 12 months following device implant to life-long administration. Most patients are followed at 3 to 6 months, and then, for 1, 2 and 3 years following device implantation (by FDA guidelines) with variable requirements for echocardiograms, chest x-rays and electrocardiograms.

In the Journal of Interventional Cardiology, Volume 19 Issue 2 Page 163-165, April 2006 an article by HENRIK TEN FREYHAUS M.D, STEPHAN ROSENKRANZ M.D, MICHAEL SÜDKAMP M.D, HANS-WILHELM HÖPP M.D (2006) entitled “Dysfunction of an Atrial Septal Defect Occluder 8 Years after Implantation Journal of Interventional Cardiology” noted that catheter interventional treatment of atrial septal defect (ASD) is widely accepted. The article goes on to suggest that the ASD occluder system (ASDOS) is no longer a widely used device nowadays, but, however, it is implanted in a substantial number of patients. The article reports a case of severe left-to-right shunt 8 years after catheter interventional closure of an ASD with an ASDOS device. The shunt was due to a membrane perforation, while the arms of the device were not dislocated. Microscopy, microbiology, and histology could not establish a proper explanation for the dysfunction; so the article concluded that long-term follow-up investigation may be required in patients with an implanted ASDOS device.

Conclusions Regarding ASD Repair

There remains a need or desire to achieve the same closure results as obtainable with surgical procedures used to patch and repair ASD, while utilizing the benefits of trans-catheter closure of the atrial septal defect. It is an object of the present invention to address the deficiencies of the prior art discussed above and to do so in an efficient, effective manner.

Patent Foramen Ovale (PFO)

The foramen ovale is a small flap located in atrial septum that is used during fetal circulation to bypass the fluid-filled fetal lungs. When in the womb, a baby does not use its own lungs for oxygen-rich blood; it relies on the mother to provide oxygen rich blood from the placenta through the umbilical cord to the fetus. Therefore, blood can travel from the veins to the right side of the baby's heart and cross to the left side of the heart through the foramen ovale.

Normally the foramen ovale closes at birth when increased blood pressure on the left side of the heart forces the opening to close. If the atrial septum does not close properly, it is called a patent foramen ovale. This type of atrial shunt generally works like a flap valve, only opening during certain conditions when there is more pressure inside the chest. This increased pressure occurs when people strain while having a bowel movement, cough, or sneeze.

If the pressure is great enough in a patient with PFO, blood may travel from the right atrium to the left atrium. If there is a clot or particles in the blood traveling in the right side of the heart, it can cross the PFO, enter the left atrium, and travel out of the heart and to the brain causing a stroke or into a coronary artery causing a heart attack.

The prevalence of PFO is about 25 percent in the general population. In patients who have stroke of unknown cause (cryptogenic stroke), the prevalence of PFO increases to about 40 percent. This is especially true in patients who have had a stroke at age less than 55 years. A PFO can be associated with atrial septal aneurysm, which is characterized by excessive mobility of the atrial septum.

Medical Management of PFO

The current standard of care indicates that patients with PFO do not need any treatment if there is no associated problems, such as a stroke. Patients who have had a stroke or transient ischemic attack (TIA) may be placed on some type of blood thinner medication, such as aspirin, plavix (clopidogrel), or coumadin (warfarin) to prevent recurrent stroke.

Non-Surgical Treatment of PFO with Cardiac Implant

In some patients a cardiologist and a neurologist may recommend closure of PFO. Most frequently, percutaneous rather than surgical closure is preferred. As part of the procedure, the patient will undergo cardiac catheterization. Currently there are no specially designed devices for PFO closure that is approved by the FDA in the United States. In patients that closure is indicated and a Non-surgical approach is selected, the ASD closure devices described above are used.

Conclusions Regarding PFO Repair

There remains a need or desire to achieve the same closure results as obtainable with surgical procedures used to repair PFO, while utilizing the benefits of trans-catheter closure of PFO. It is an object of the present invention to address the deficiencies of the prior art discussed above and to do so in an efficient, effective manner. Within the meaning of this application ASD and PFO are both Septal Defects.

Left Atrial Appendage Closure

Atrial fibrillation is a relatively common cardiac rhythm disorder affecting a population of approximately 2.5 million patients in the United States alone. Atrial fibrillation results from a number of different causes and is characterized by a rapid chaotic heart beat. During this type of fibrillation, the atria, rather than the sinus node, initiates the impulses which cause contraction of the heart muscle. In some patients, atrial fibrillation may occur in the absence of any other known disease. These impulses are relatively rapid and erratic, and are known to not properly control the contractions of the heart. As a result, the atria beat faster than the ventricles, the ventricular contractions are irregular, the ventricles do not completely fill, with blood, and the ventricular contractions eject less blood into the greater vessels.

The atrial appendages are especially important in the transport of blood because they have a sack-like geometry with a neck potentially more narrow than the pouch. In this case, contraction of the appendage is essential to maintain an average absolute blood velocity high enough to eliminate potential stasis regions which may lead to thrombus formation. One of the many problems caused by atrial fibrillation is the pooling of blood in the left atrial appendage during fibrillation. When blood pools in the atrial appendage, blood clots can accumulate therein, build upon themselves, and propagate out from the atrial appendage into the atrium. These blood clots can cause serious problems when the heart resumes proper operation (normal sinus rhythm) and the blood, along with the blood clot(s), is forced out of the left atrial appendage. Similar problems also occur when a blood clot extending from an atrial appendage into an atrium breaks off and enters the blood supply. More specifically, the blood from the left atrium and ventricle supply the heart and brain. Thus, the blood flow will move the clots into the arteries of the brain and heart which may cause an obstruction in blood flow resulting in a stroke or heart attack. Consequently, patients with atrial fibrillation also have an increased risk of stroke. It has been estimated that approximately 75,000 atrial fibrillation patients each year suffer a stroke related to that condition.

Significant efforts have been made to reduce the risk of stroke in patients suffering from atrial fibrillation. Most commonly, those patients are treated with blood thinning agents, such as warfarin, to reduce the risk of clot formation. While such treatment can significantly reduce the risk of stroke, it also increases the risk of bleeding and for that reason is inappropriate for many atrial fibrillation patients.

An alternative to the drug therapy is a procedure that closes (stitch off or remove) the left atrial appendage in patients which are prone to atrial fibrillation. Most commonly, the left atrial appendage has been closed or removed in open surgical procedures, typically where the heart has stopped and the chest opened through the sternum. Because of the significant risk and trauma of such procedures, left atrial appendage removal occurs almost exclusively when the patient's chest is opened for other procedures, such as coronary artery bypass or valve surgery.

For that reason, alternative procedures which do not require opening of the patient's chest, i.e., a large median sternotomy, have been proposed. U.S. Pat. No. 5,306,234 to Johnson describes a thoracoscopic procedure where access to the pericardial space over the heart is achieved using a pair of intercostal penetrations (i.e., penetrations between the patients ribs) to establish both visual and surgical access. While such procedures may be performed while the heart remains beating, they still require deflation of the patient's lung and further require that the patient be placed under full anesthesia. Furthermore, placement of a chest tube is typically required to re-inflate the lung, often requiring a hospitalization for a couple of days.

U.S. Pat. No. 5,865,791, to Whayne et al. describes a transvascular approach for closing the left atrial appendage. Access is gained via the venous system, typically through a femoral vein, a right internal jugular vein, or a subclavian vein, where a catheter is advanced in an antegrade direction to the right atrium. The intra-atrial septum is then penetrated, and the catheter passed into the left atrium. The catheter is then positioned in the vicinity of the left atrial appendage which is then fused closed, e.g., using radiofrequency energy, other electrical energy, thermal energy, surgical adhesives, or the like. Whayne et al. further describes a thoracoscopic procedure where the pericardium is penetrated through the rib cage and a lasso placed to tie off the neck of the left atrial appendage. Other fixation means described include sutures, staples, shape memory wires, biocompatible adhesives, tissue ablation, and the like. The transvascular approach suggested by Whayne et al. is advantageous in that it avoids the need to penetrate the patient's chest but may not provide definitive closure and requires injury to the endocardial surface which may promote thrombus formation. A thoracoscopic approach which is also suggested by Whayne et al. suffers from the same problems as the thoracoscopic approach suggested by Johnson.

Conclusions Regarding Left Atrial Appendage Closure

There is a need for an acceptable tool for minimally invasive left atrial appendage closure. It would be further desirable to provide an effective, efficient, easily utilized tool that allows for procedures which approach the left atrial appendage without the need to perform a thoracotomy (i.e. penetration through the intercostal space) or the need to perform a transeptal penetration and/or perform the procedure within the left atrium or left atrial appendage. At least some of these objectives will be met by the invention described herein below.

Mitral Valve Repair

Mitral valve repair is a cardiac surgery procedure performed to treat stenosis (narrowing) or regurgitation (leakage) of the mitral valve. The mitral valve is the “inflow valve” for the left side of the heart. Blood flows from the lungs, where it picks up oxygen, through the pulmonary veins, to the left atrium of the heart. After the left atrium fills with blood, the mitral valve allows blood to flow from the left atrium into the heart's main pumping chamber called the left ventricle. It then closes to keep blood from leaking back into the left atrium or lungs when the ventricle contracts (squeezes) to push blood out to the body. It has two flaps, or leaflets. Procedures on the mitral valve usually require a median sternotomy, but advances in non-invasive methods, such as keyhole surgery allow surgery without a sternotomy.

In 1923 Dr. Elliot Cutler performed the world's first successful heart valve surgery, a mitral valve repair on a 12-year-old girl with rheumatic mitral stenosis. The development of the heart-lung machine in the 1950s paved the way for replacement of the mitral valve with an artificial valve in the 1960s. For decades, mitral valve replacement was the only surgical option for patients with a severely diseased mitral valve. In the last two decades, some surgeons have embraced surgical techniques to repair the damaged native valve, rather than replace it. These techniques have been attributed to a French heart surgeon, Dr. Alain F. Carpentier. A repair still involves major cardiac surgery but for many patients presents the major advantage of avoiding blood thinners and may provide a more durable result. Not all damaged valves are suitable for repair—in some the state of valve disease is too advanced and replacement is necessary. Often a surgeon can only make a decision of repair versus replace during the actual operation. Within the meaning of this application the term mitral valve repair is intended to include a repair of the valve and a replacement of the valve.

There has been great debate about timing of surgery in patients with asymptomtic mitral valve regurgitation. There are minimally invasive (port access) options available pioneered by Hugo Vanerman in Belgium. They allow a safe way to repair the mitral valve and allow the patient to return to their normal activity much sooner than the standard approach. In the last decade there have been several trials of a newer strategy of mitral valve repair that does not require major cardiac surgery. Through a catheter inserted in the groin the valve leaflets are clipped together. Since the early 1990's, edge-to-edge has been increasingly used in the treatment of mitral regurgitation (MR). Pioneered in Italy by Dr. Ottavio Alfieri, the technique involves suturing together the two leaflets of the mitral valve. The valve continues to open on both sides of the suture, allowing blood flow through the valve from the left atrium to left ventricle, while assuring proper valve closure when blood is pumped from the left ventricle to the rest of the body. The Evalve Percutaneous Mitral Repair (PMR) system from Evalve, Inc. is intended to reduce mitral regurgitation (MR), adapting the open surgical edge-to-edge technique in a less invasive catheter-based procedure. The Evalve Percutaneous Mitral Repair (PMR) system relies upon a clip type fastener to secure the edges together.

Conclusions Regarding Mitral Valve Repair

There remains a need or desire to achieve the same closure results as obtainable with surgical procedures used to repair the mitral valve, while utilizing the benefits of trans-catheter repair of the mitral valve. It is an object of the present invention to address the deficiencies of the prior art discussed above and to do so in an efficient, effective manner.

Pacemaker Electrode Placement

A pacemaker, also called an artificial pacemaker, so as not to be confused with the heart's natural pacemaker, is a medical device which uses electrical impulses, delivered by electrodes contacting the heart muscles, to regulate the beating of the heart. The primary purpose of a pacemaker is to maintain an adequate heart rate, either because the heart's native pacemaker is not fast enough, or there is a block in the heart's electrical conduction system. Modern pacemakers are externally programmable and allow the cardiologist to select the optimum pacing modes for individual patients. Some combine a pacemaker and implantable defibrillator in a single implantable device. Others have multiple electrodes stimulating differing positions within the heart to improve synchronization of the lower chambers of the heart.

Permanent pacing with an implantable pacemaker involves transvenous placement of one or more pacing electrodes within a chamber, or chambers, of the heart. The procedure is performed by incision of a suitable vein into which the electrode lead is inserted and passed along the vein, through the valve of the heart, until positioned in the chamber. The procedure is facilitated by fluoroscopy which enables the physician or cardiologist to view the passage of the electrode lead. After satisfactory lodgment of the electrode is confirmed the opposite end of the electrode lead is connected to the pacemaker generator.

Conclusions Regarding Electrode Placement

There is a need for a tool for minimally invasive electrode placement and secure anchoring of the electrode. It would be further desirable to provide an effective, efficient, easily utilized surgical tool that allows for secure electrode placement.

Cardiac Ablation Catheter Positioning

A cardiac ablation catheter is a medical device which is used to detect and correct abnormal electrical pathways in heart tissue. Abnormal electrical activity in the heart tissue causes cardiac arrhythmias such as atrial fibrillation, which if left untreated can be harmful or life threatening as previously described. A catheter with distal electrodes is articulated and placed against the internal walls of the heart and “maps” or detects the local electrical activity. The catheter position may be adjusted until a region of abnormal electrical activity is detected. Once detected, the responsible tissue is eliminated or scarred in order to inhibit the abnormal electrical pathway. Tissue scarring is effected using either radio frequency or microwave energy emitted from the distal electrodes, or by freezing the local tissue by flowing an extremely low temperature coolant through the distal tip of the catheter (cryoablation).

Conclusions Regarding Cardiac Ablation Catheter Positioning

There is a need for accurate and stable placement of ablation catheters against localized regions of the internal myocardial walls. It would be further desirable to provide some assurance that an ablation catheter position does not shift between the time of mapping a region of abnormal electrical activity and tissue ablation. It is the object of this

### SUMMARY OF THE INVENTION

The various embodiments and examples of the present invention as presented herein are understood to be illustrative of the present invention and not restrictive thereof and are non-limiting with respect to the scope of the invention.

According to one non-limiting embodiment of the present invention, a method of non-invasive transcatheter atrial septal defect repair comprising the steps of: advancing a positioning member along a catheter into the Atrial Septal Defect, wherein at least one suture deploying lumen is coupled to the positioning member with a piercing member within the suture deploying lumen; deploying the positioning member within the Atrial Septal Defect to align each suture deploying lumen with tissue adjacent the Atrial Septal Defect; and piercing the tissue adjacent the Atrial Septal Defect with the piercing member to secure a suture line through the tissue. A repair patch may be advanced along suture lines to repair the defect and secured into place with the suture lines.

According to one non-limiting embodiment of the present invention, an apparatus for non-invasive atrial septal defect repair comprises a positioning member configured to be moved along a catheter to the Atrial Septal Defect received and deployed within the Atrial Septal Defect; at least one suture deploying lumen coupled to the positioning member and configured to be aligned with tissue adjacent the Atrial Septal Defect when the positioning member is received and deployed within the Atrial Septal Defect; and a piercing member within the suture deploying lumen and configured to pierce the tissue adjacent the Atrial Septal Defect when the positioning member is received and deployed within the Atrial Septal Defect to secure a suture line through the tissue.

In one non-limiting embodiment of the present invention the positioning member is an expanding/contracting inflatable/deflatable member and is configured to expand when positioned within the Atrial Septal Defect. The inflatable positioning member may have a smaller diameter in the deployed position at the defect than the diameter of the positioning member in the deployed position at the location that the lumen is coupled there to.

In one non-limiting embodiment of the present invention the apparatus further includes at least one, and possibly one for each suture line, central lumen configured to extend through the Atrial Septal Defect, and wherein the at least one central lumen is configured to co-operate with the suture deploying lumen when the positioning member is received and deployed within the Atrial Septal Defect to secure a suture line through the tissue.

In one non-limiting embodiment of the present invention a plurality of suture deploying lumens are coupled to the positioning member at radial spaced positions about the positioning member.

In one non-limiting embodiment of the present invention the suture line is configured to include an expanding suture anchor adapted to prevent the suture line from being drawn back through the tissue.

According to one non-limiting embodiment of the present invention, a suture application apparatus for tissue defect repair comprises an expandable positioning member configured to be received and deployed within the tissue defect; at least one suture deploying lumen coupled to the positioning member and configured to be aligned with tissue adjacent the tissue defect when the positioning member is received and deployed within the tissue defect; and a piercing member within the suture deploying lumen and configured to pierce the tissue adjacent the tissue defect when the positioning member is received and deployed within the tissue defect to secure a suture line through the tissue.

These and other advantages of the present invention will be clarified in the description of the preferred embodiments taken together with the attached figures.

### BRIEF DESCRIPTION OF THE DRAWINGS

FIG. 1A is a schematic representation of a noninvasive trans-catheter septal defect repair device according to one non limiting embodiment of the present invention;

FIGS. 1B-1D are enlarged views of the noninvasive trans-catheter septal defect repair device of FIG. 1A;

FIGS. 2A-2N are sequential schematic sketches illustrating a method of noninvasive trans-catheter remote suture placement with the device of FIG. 1A;

FIG. 3 is an enlarged view of the hook and loop capture of FIG. 2H;

FIGS. 4A-4F are sequential schematic sketches illustrating a method of patch placement for septal defect repair following remote suture placement with the device of FIG. 1A;

FIGS. 5A-5E are sequential schematic sketches illustrating a method of patch attachment and suture trimming for septal defect repair following patch placement of FIG. 4F;

FIG. 6 is a schematic illustration of the patch repair of a septal defect with the device of FIG. 1A;

FIG. 7 is an enlarged view of a hook and suture coupling for the device of FIG. 1A;

FIGS. 8A and 8B schematically illustrate an anchored suture embodiment of the present invention which is one modification of the device of FIG. 1A;

FIGS. 9A and 9B schematically illustrate a second anchored suture embodiment of the present invention which is one modification of the device of FIG. 1A; and

FIGS. 10A and 10B schematically illustrate a third anchored suture embodiment of the present invention which is one modification of the device of FIG. 1A.

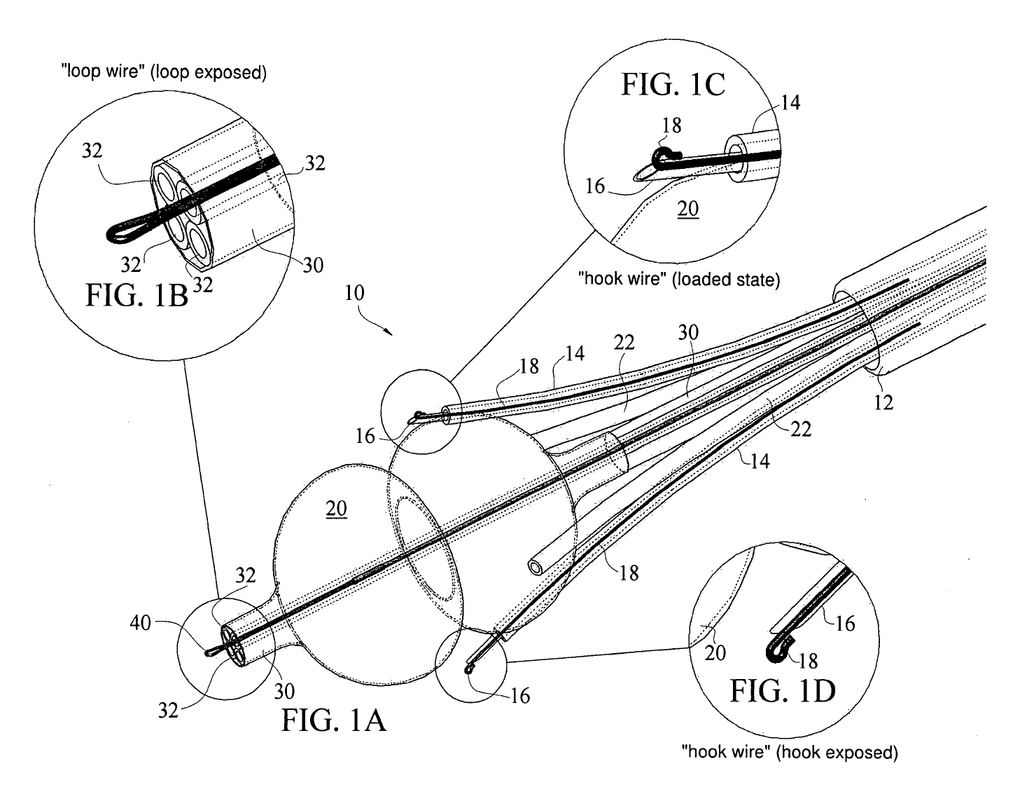

### DESCRIPTION OF THE PREFERRED EMBODIMENTS

Non-Invasive Trans-Catheter Septal Defect Repair Device 10

According to one non-limiting embodiment of the present invention, a summary or overview of a method of non-invasive transcatheter atrial septal defect repair comprises the steps of: advancing a positioning member 20 which is part of device 10 along a catheter 12 into the Atrial Septal Defect 7 of tissue 5, wherein at least one suture deploying lumen 14 is coupled to the positioning member 20 with a piercing member 16 within the suture deploying lumen 14; deploying the positioning member 20 within the Atrial Septal Defect 7 to align each suture deploying lumen 14 within close axial proximity to tissue 5 just adjacent to the Atrial Septal Defect 7; facilitating puncture of the tissue 5 adjacent the Atrial Septal Defect with the piercing member 16 to secure a suture line 50 through the tissue 5. A repair patch 90 may be advanced along suture lines 50 to repair the defect 7 and secured into place using an appropriate slip knot 56 fastened in the suture lines 50. The following description will provide a more detailed explanation of the method and apparatus of the present invention.

The device 10 according to the present invention is intended for trans-catheter application as shown in the figures. The device 10 may be sized in accordance with the specific catheter 12 being utilized. The construction of catheter 12 is well known in the art of heart catheterization, and these teachings are incorporated herein by reference.

The device 10 of the present invention includes a plurality of flexible lumens 14 substantially spaced at even radial positions about the positioning member 20 and moveable along or through the catheter 12. The construction of the lumens 14 may be from any material that is sufficiently flexible to be moved within a heart catheter 12 and can accommodate the outward flexing as shown below. Further, the disclosure illustrates four lumens 14, but this is merely for illustrative purposes. The precise number and spacing of lumens 14 can be left to the particular application. Four lumens 14 are shown as this is illustrative of placing a plurality of sutures around the periphery of a defect 7 in the tissue 5. It is anticipated that the larger the defect 7 the larger the number of lumens 14. Regarding the spacing, an even radial spacing between adjacent lumens 14 seems the most practical to evenly space delivered sutures 50 about the defect 7, but the operation of the device 10 is unchanged if there is an uneven spacing between the lumens 14. Further, for certain applications, such as the application of a single electrical lead, only one lumen 14 may be provided in the device 10.

The device 10 of the present invention provides a piercing member 16 within each of the lumens 14 and which is axially moveable relative to the associated lumen 14 as discussed in detail below. The construction of the majority of the piercing member 16 may be from any material that is sufficiently flexible to be moved within a heart catheter 12 and can accommodate the outward flexing as shown below, generally similar in requirement to the lumen 14. However, the piercing member 16 must further have a construction at the distal end thereof that allows the piercing member 16 to pierce the tissue 5 for suture 50 application as will be described below. This may be accomplished with an angular beveled edge on the material forming the piercing member 16 as shown. Alternatively, a separate piercing tip, e.g. a metal end, can be provided at the distal end of the piercing member 16 such that the end is a different harder material than the remainder of the member 16.

The device 10 of the present invention provides a memory wire 18, with a grasping hook formed at the end thereof, provided within each of the piercing members 16. The wire 18 is axially and rotationally moveable relative to the associated piercing member 16 as discussed in detail below. Nitinol is a known shape memory alloy that is well suited for forming the wire 18. Other shape memory alloys may also be utilized. It is important for the present invention that the wire 18 return to an arcuate or curved shape as it extends from the piercing member 16 in order to facilitate grasping of the wire 1 8 on the other side of the tissue 5. The length of the wire 18 should be slightly longer than the catheter 12 for a length sufficient to allow for user control of the wire 18 through out the deployment and wire grasping operation described below. The distal end of the wire 18 is formed in the shape of a grasping hook as shown. The proximal end of the wire 18 is attached to the suture line 50, also called the suture 50. A simple crimping attachment 52 can secure the wire 1 8 to the suture line 50, which needs to be a sufficiently secure attachment to allow a pulling of the wire 18 and suture 50 through the device 10 (distally through lumen 14 and proximally back through central lumen 32) to accomplish the full deployment (i.e. pulling) of one suture 50.

The device 10 of the present invention provides an expanding positioning member 20 to which the lumens 14 are coupled. The positioning member 20, as shown is an inflatable member with a cinched waist for properly positioning the lumens 14 in close axial proximity to tissue 5 and adjacent to the defect 7. A centrally located inflation tube (not shown) allows the positioning member 20 to be expanded and contracted. As described below the “cinched waist” shape of the positioning member 20 in the deployed position will properly position the device 10 axially relative to the tissue 5 and defect 7 and will properly position the lumens radially relative to the defect 7. The construction of the inflatable member 20 can be substantially similar to angioplasty devices, except with a cinched waist configuration. Other expanding shapes can be possible other than an inflating member. Functionally the member 20 needs to be (1) substantially freely passable through the catheter 12 (with lumens 14) in the collapsed position, (2) substantially freely passable through the defect 12 at least in the collapsed position, (3) expandable within the defect 7 to position the lumens 14 at tissue 5 adjacent to the defect 7, and (4) substantially collapsible to facilitate removal from defect 7 following suture 50 deployment. The double bulb shaped inflatable member 20 is one way of providing the needed functionality.

The positioning member 20 is provided on a central member 30 or core 30 which also houses central lumens 32. The device 10 includes a plurality of central lumens 32 that equal the number of lumens 14 on the device. Each central lumen 32 houses an extendable memory grasping loop 40 that is extendable from the central lumen 32. The loop 40 is axially and rotationally moveable relative to the associated central lumen 32 as discussed in detail below. Nitinol is a known shape memory alloy that is well suited for forming both the wire 18 and the loop 40. Other shape memory alloys may also be utilized. It is important for the present invention that the loop 40 will assume an arcuate or curved shape as it extends from the lumen 32 in order to facilitate grasping of the wire 18 on the other side of the tissue 5. The length of the loop 40 should be slightly longer than the catheter 12 for a length sufficient to allow for user control of the loop 40 through out the deployment and wire grasping operation described below. The distal end of the loop 40 is formed in the shape of an elongated target loop as shown. The proximal end of the loop 40 may be any form that is easy for the operator to grasp and manipulate.

Method of Non-Invasive Trans-Catheter Suture 50 Placement using the Septal Defect Repair Device 10

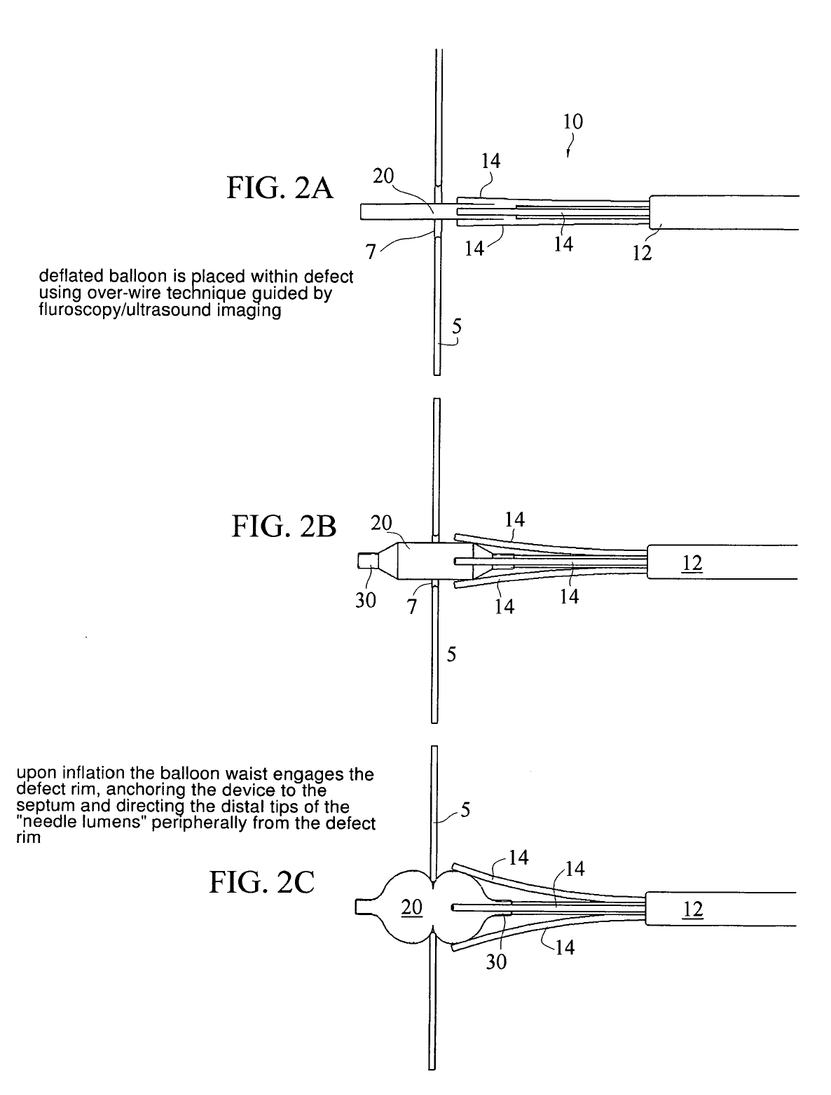

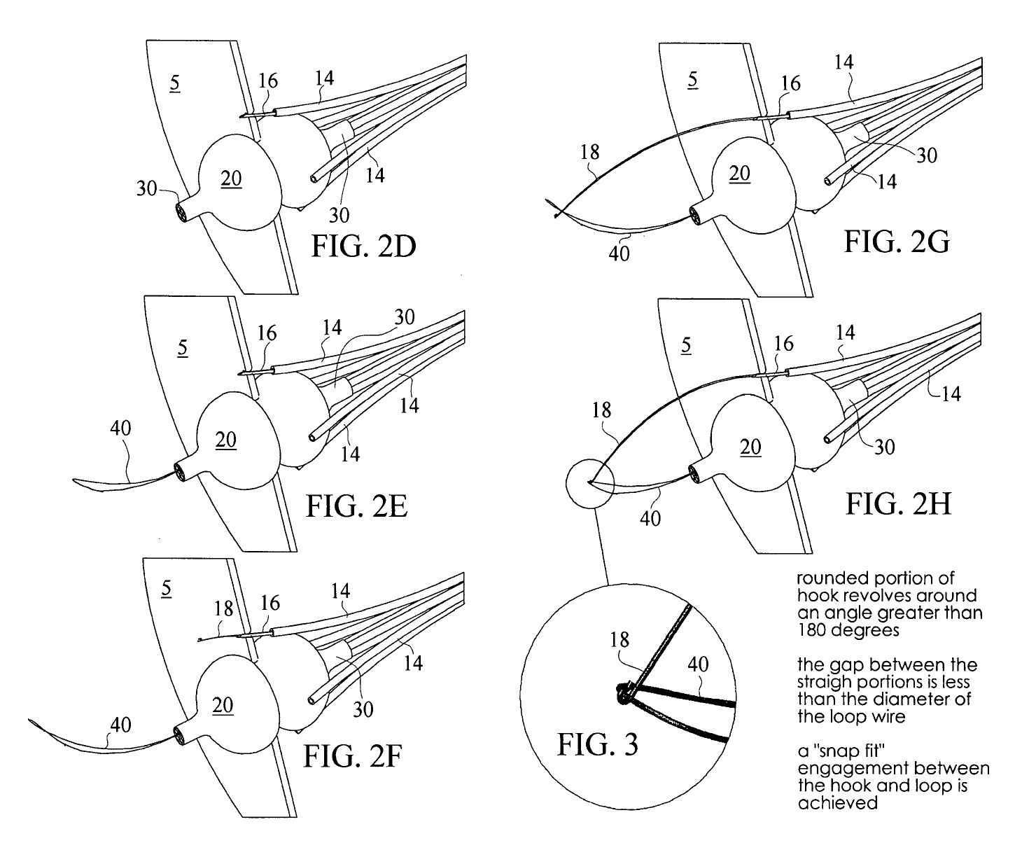

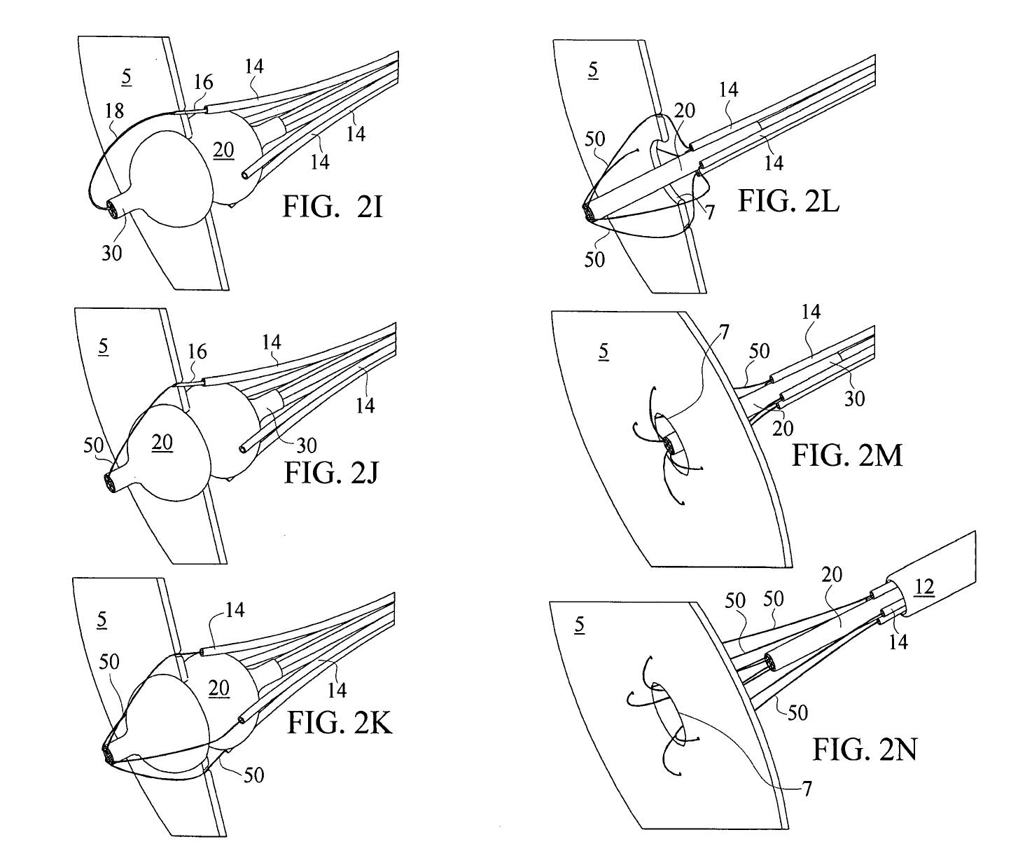

The advantages of the device 10 in accordance with the present invention will be further clarified in a description of the remote non-invasive trans-catheter suture 50 placement using the septal defect repair device 10 shown in FIGS. 2A-2N. The first step is working a catheter 12 to a position, such as represented in FIG. 2A, adjacent the defect 7 in the tissue 5. This process is believed to be well known in the heart catheterization fields and is not described herein in detail. The defect 7 of the figures is representative of an atrial septal defect (ASD) and the tissue 5 is the septum, but obviously this can be representative of other tissue defects.

Following the placement of the catheter 12 to a position adjacent the defect 7 the device 10 is advanced through the catheter 12 with the positioning member 20 in the collapsed or retracted position, and the positioning member is advanced within the defect 7 as shown in FIG. 2A. This positioning may be assisted with fluoroscopic or ultrasonic imaging and may utilize an over-wire technique with the core 30 receiving the guide wire (not shown). Similar over wire positioning is used for occlusion implant placement in ASD repair discussed above.

FIGS. 2B and 2C illustrate the expansion of the positioning member 20 once it has been properly deployed. The cinched waist design of the member 20 will anchor the device 10 to the tissue 5 in the proper location within the septum or tissue 5. Furthermore, the expansion of the positioning member 20 will direct the lumens 14 to the tissue surrounding the defect 7. The cinched waist of the member 20 may be expanded to slightly larger than the defect 7 to accommodate defects that are not precisely round. This raises an important aspect of the present invention. The member 20 is designed for the particular desired application, such as atrial septal defect. If it is found that such defects ordinarily have an oblong or oval shape then the member 20 can be formed accordingly having a similar shape (still with a cinched waist). In such designs, imaging could be used to properly orient the device 10 rotationally within the defect 7. The illustrated embodiment uses a round shape for the member 20 and therefore no orientation is required. As such the round shape is preferred.

FIG. 2D illustrates the advancement of one piercing member 16 from the lumen 14 though the tissue 5. The expansion of the member 20 aligns the lumen 14 such that the piercing member 16 punctures the tissue 5 in a proper location adjacent to the defect 7. FIGS. 2E-2G illustrate the advancement of one of the loops 40 from one of the central lumens 32 and the advancement of the wire 18 from the piercing member 16 to a point where the wire is through the loop 40 as shown. This is where the arcuate or curved shape assumed by both the loop 40 and the wire 18 is useful and beneficial. This positioning may be assisted with fluoroscopic or ultrasonic imaging. Rotating the respective wires 18 and loops 40 by the operator can adjust the relative position to achieve the wire 18 in loop 40 orientation of FIG. 2G.

After the wire 18 is through the loop 40 the loop 40 can be retracted until the loop 40 engages and captures the hook at the end of the wire 18. The gap at the end of the hook on wire 18 may be less than the diameter of the loop forming wire of loop 40 to provide a snap fit engagement. An enlarged view of this positive engagement is shown in FIG. 3.

Following the capture of the wire 18 by the loop 40, the loop 40 is retracted through the central lumen 32 drawing the wire 18 into the central lumen 32 as represented in FIG. 2I. The operator will continue to withdraw the loop 40 then the wire 18 until the suture 50 is pulled entirely through the piercing member 16 through the tissue 5 and back through the central lumen 32 as represented in FIG. 2J. Once the suture line 50 is retrieved from the proximal end of the central lumen 32, it may be cut to remove the wire 50 and coupler 52 (and the attached loop 40).

The above procedures is repeated for each suture line 50 (i.e. each lumen 14 and each central lumen 32), until all sutures 50 are in place as shown in FIG. 2K. Following application of the sutures 50, the positioning member 20 is collapsed and withdrawn through the catheter 12 as shown in FIGS. 2L-2N. The suture lines 50 are left behind, which is the entire point of the suture application of device 10.

The above description results in a plurality of through suture lines 50 extending into the tissue 5 adjacent the defect 7 and back through the defect 7. FIGS. 8-10 illustrate a separate suture 50 delivery methodology in which an anchor 60, 70 or 80 is deployed into or through the tissue 5 to hold the suture 50 about the defect 7 rather than having it retrieved through the defect 7. These suture delivery methodologies do not require the use of the central lumens 32, the wire 18, the loop 40, or the associated wire engagement methodology described above. In each of these embodiments, following the piercing of the tissue with the piercing member 16 in FIG. 2D, also represented in FIGS. 8A, 9A and 10A, respectively, the suture 50 and associated anchor 60, 70 or 80 is advanced through the tissue 5. The respective attached anchor 60, 70 or 80 will deploy to an anchoring position as shown once it is moved beyond the distal tip of piercing member 16. The illustrated embodiments are intended to demonstrate the wide variety of tissue anchors that may be possible. Further, if the suture line 50 is insufficient in strength to be advanced along the piercing member 16 remotely a separate pushing rod (not shown) could be used. The end result is an anchored suture 50 that may be acceptable for a variety of applications. Following deployment of the anchored sutures 50 as shown in FIGS. 8B, 9B and 10B respectively, the positioning member 20 is retracted and the device 10 removed substantially as shown in FIGS. 2L-2N described above (except that each suture 50 will have only a single extension from the tissue 5 rather than a lead and return extension as shown in FIGS. 2L-2N).

Method of Non-Invasive Trans-Catheter Patch Placement using Sutures 50 Delivered with the Septal Defect Repair Device 10

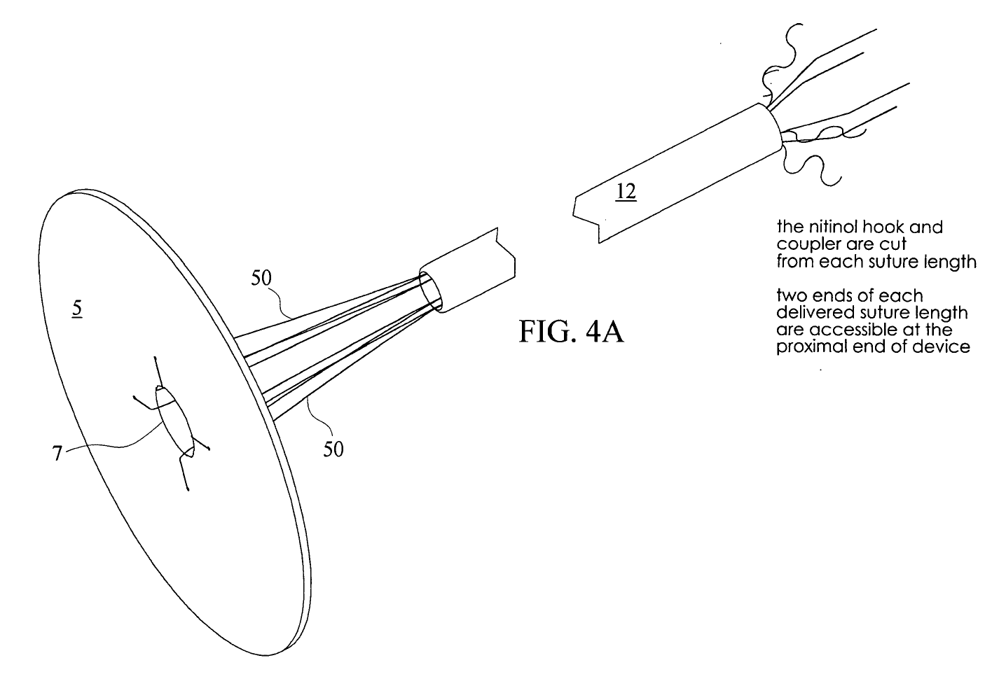

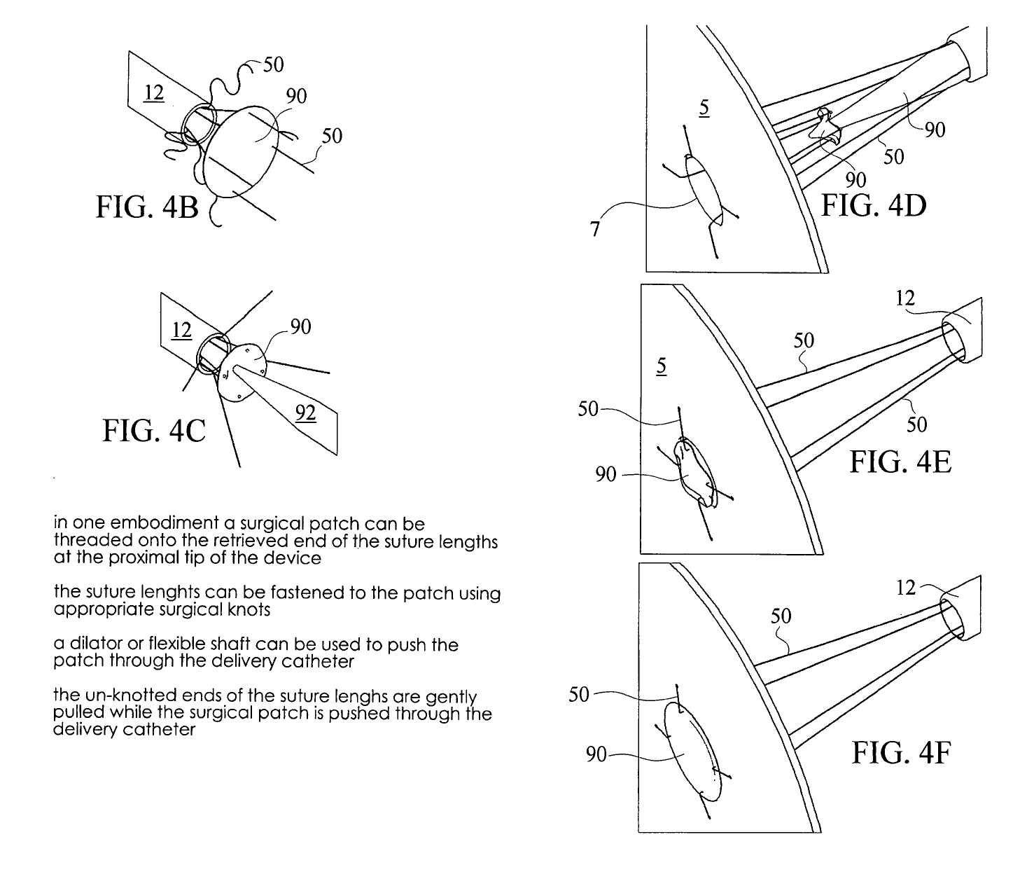

With the sutures 50 in place as described above and shown in FIGS. 4A a patch 90 can be guided through the catheter 12 to the proper location. With the sutures 50 delivered through the tissue 5, one end of each suture 50 can be secured to the patch 90 at radial spaced positions about the patch 90 as shown in FIGS. 4B and 4C. This attachment shows one of the benefits of separate central lumens 32. The use of central lumens 32 prevents undesirable twisting or tangling of the sutures 50 together within the catheter 12 which would make the application of the patch 90 very difficult if not impossible due to the level of inadvertent twisting or tangling.

The tied patch 90 can be advanced through the catheter 12 by pulling on the free ends of the sutures 50, and with the assistance of a pusher 92. Once in position at the defect as shown in FIG. 4E, taught pulling of the sutures 50 will firmly position the patch 90 across the defect 7.

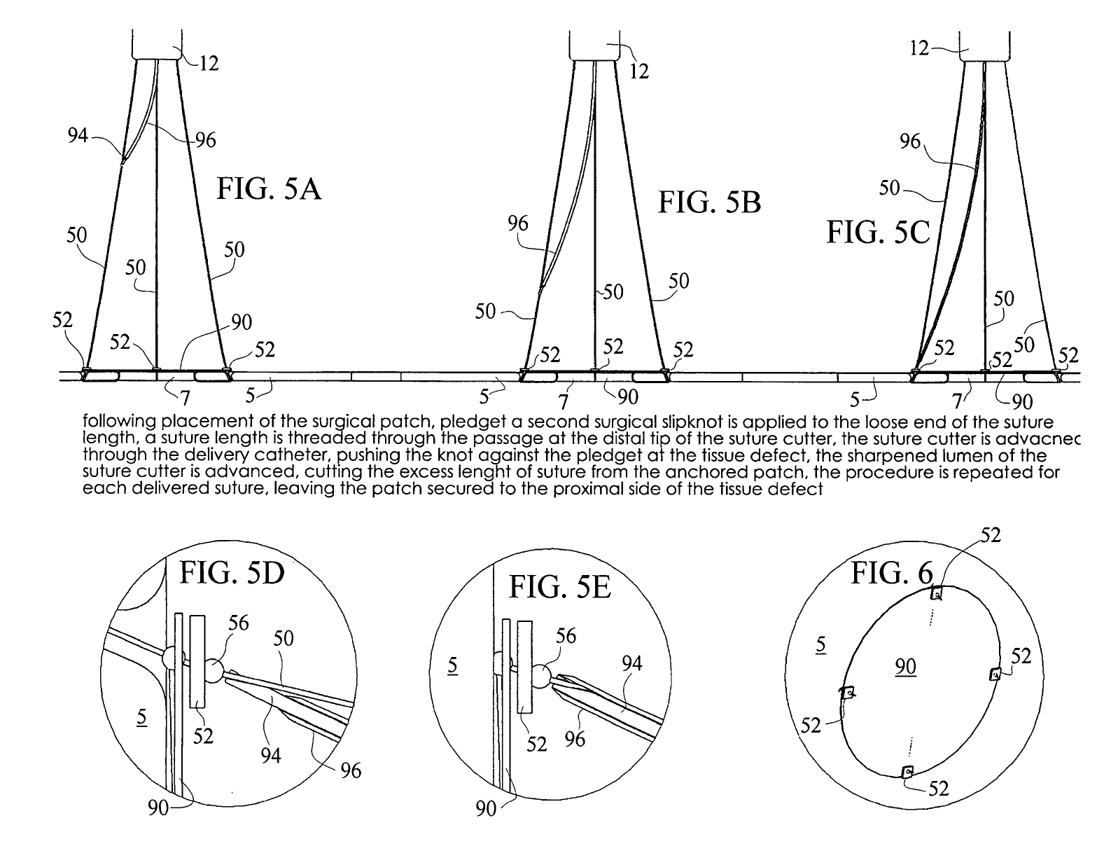

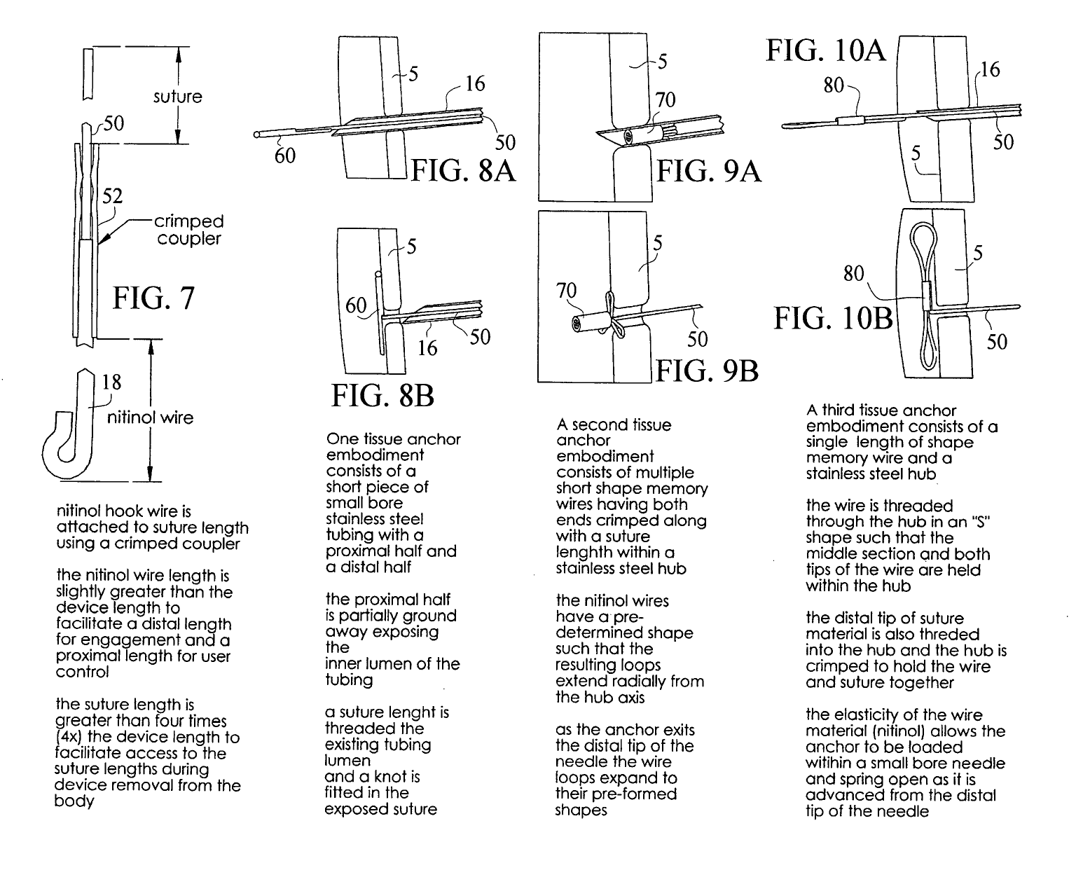

For the embodiments that use an anchored line as illustrated in FIGS. 8-10, a pusher 92 will be the mechanism for advancing the patch 90 and for pushing it into the final patch position across the defect 7. The patch 90 will end on the proximal side of the defect rather than on the distal side, but this is not believed to have a substantive effect on defect closure and FIGS. 5A-5E illustrate this orientation of the patch and defect.

FIGS. 5A-5E illustrate the securing of the patch 90 and trimming of the sutures 50 to finally secure the patch 90 in position. The initial step is advancing holding members 52 to a position adjacent the tissue 5 or the patch 90, as schematically shown in FIG. 5A. The members 52 may be considered or formed as pledgets. A slip knot 56 is formed in each suture 50 and advanced with a knot pusher comprised of a pusher rod 94 fitted with a distal side port and severing member 96 in the form of a sharpened outer sheath as shown in FIGS. 5B and 5C. Once each slip knot 56 snuggly holds the associated patch 90 in place, the excess line 50 may be trimmed by the shearing action of the pusher rod 94 distal side port and the distal sharpened portion of the severing member 96 as shown in FIGS. 5D and 5E and the excess line 50 and the elements 94 and 96 removed with the catheter 12. The final patched defect 7 is illustrated schematically in FIG. 6.

Patent Foramen Ovale (PFO) and Non Patch Repair of Septal Defect

The patch repair of tissue defects described above will be effective for repair of atrial septal defects (ASD) as described and for repair of patent Foramen Ovale (PFO) without any substantive modifications to the device 10 or its operation. Furthermore, in the case of PFO repair, the sutures 50 alone may be used without a patch 90, or other occlusion device. The sutures 50 may be used to proximate the tissue 5 together across the defect 7 in a more natural tissue repair construction using sutures 50 alone.

For example PFO is a septal defect in which a substantial flap of tissue exists which can extend over some or all of the tissue defect 7. In such a case the device 10 of the present invention might be effectively used for tissue repair using sutures 50 alone. Specifically, the retrieved ends of the suture lines 50, if retrieved through a common central lumen 32 can be attached together and pulled back through by the proximal ends until the attached sutures will extend across the defect 7 holding the flap closed against the septum or tissue 5. For this application the multitude of separate central lumens 32 would be replaced with a single common central lumen. In this closure method no separate patch is required. Other pure tissue repair can be used. All of the retrieved ends can be attached together to form a collection of cross sutures holding the flap in place to close the PFO defect.

Left Atrial Appendage Closure

The device 10 can effectively be used for non-invasive trans-catheter left atrial appendage (LAA) closure without any substantive modifications to the device 10 or its operation. In this application the target closure orifice is the opening between the left atrium (LA) and the LAA rather than a shunt between the left and right atria. In this case, the anchored sutures of FIGS. 8-10 would be preferred as the left atrial appendage is a smaller cavity offering less room for suture retrieval. For large LAA openings a patch 90 closure would be most effective, while for smaller LLA openings tissue proximation though use of the sutures 50 may be possible and may be preferred.

Mitral Valve Repair

The device 10 can effectively be used to aid in minimally invasive mitral valve replacement or repair procedures. In the case of replacement procedures the sutures 50 such as shown in FIGS. 4A can be used as placement guides and attachment mechanism for guiding and securing a replacement valve (in place of patch 90).

In the case of repair procedures the sutures 50 can be used to pull together and selectively reshape the annulus of the mitral valve anatomy similar to the tissue approximation discussed above with the goal of improving the operating efficiency of the existing valve leaflets. The reshaping can take any number of specific final configurations depending upon which areas of the mitral valve anatomy are selectively pulled together. The relative position of suture introduction points, and hence reshaping strategies, can be employed through various spacing configurations of the lumens 14 about the positioning member 20.

Pacemaker Electrode Placement

The device 10 can effectively be used for trans-catheter pacemaker electrode placement. In using the device 10 for pacemaker electrode placement the suture lines 50 are replaced with the pacer electrodes or leads and the existing lead attachment mechanisms. In this application the positioning member 20 is not received within a defect in the tissue, as such, the positioning member 20 will be shaped to conform to the heart chamber anatomy local to the lead placement target, and the position of lumen(s) 14 might be modified according to target lead placement. For example, a common target for lead placement is in the apex of the right ventricle. In this case, the positioning member 20 might take on a conical shape, and the lumen 14 position might be moved from the periphery of the positioning member 20 to its center, replacing the central lumen(s) 32.

Further, in this application an expanding mesh frame may be preferable to an inflatable structure as an expanding mesh or frame structure will minimize blood flow displacement or blockage during the procedure. This described alternate embodiment might allow for repeatable and accurately positioning of pacer leads from the lumens 14 in the apex of the right ventricle or other pacer lead targets. The precision in the lead placement will both be relative to the heart anatomy, but also relative spacing from each other where multiple leads are utilized in a local targeted area. This application will minimize the time for lead placement and maximize consistency in lead placement positioning using only ultrasound or fluoroscopic imaging without the requirement for precision steering or navigational technologies.

Cardiac Ablation Catheter Placement

The device 10 through minor modification can effectively be used for positioning cardiac ablation catheters. In this application, piercing member(s) 16, suture(s) 50, wire(s) 18, and loop(s) 40 might be eliminated, and lumen(s) 14 might be replaced with a cardiac ablation catheter which is well known in the field. In this application expanding mesh frame may be a preferable configuration for the positioning member 20 versus inflatable structure as an expanding mesh or frame structure will minimize blood flow displacement or blockage during the procedure. Furthermore, positioning member 20 might take on the shape of the internal heart chambers in the expanded state so as to press the attached cardiac ablation catheter firmly against the internal myocardial walls. In this manner, a cardiac ablation catheter can be firmly positioned in an area of the heart so that the area can be electrically mapped to locate abnormal electrical activity, and by virtue of the positioning member 20, shifting of the catheter prior to tissue ablation is unlikely. Furthermore, this can be achieved without the need for precision catheter articulation or navigation technology.

Whereas particular embodiments of the invention have been described above for purposes of illustration, it will be evident to those skilled in the art that numerous variations of the details of the present invention may be made without departing from the spirit and scope of the present invention. For example, instead of a single cinched waist chamber forming inflatable expanding member 20, the member 20 may be formed of independently inflatable segments, that form the same overall shape.

What is claimed is:

1. An apparatus for non-invasive atrial septal defect repair comprising:
A positioning member configured to be moved along a catheter to the Atrial Septal Defect received and deployed within the Atrial Septal Defect,
At least one suture deploying lumen coupled to the positioning member and configured to be aligned with tissue adjacent the Atrial Septal Defect when the positioning member is received and deployed within the Atrial Septal Defect; and
A piercing member within the suture deploying lumen and configured to pierce the tissue adjacent the Atrial Septal Defect when the positioning member is received and deployed within the Atrial Septal Defect to secure a suture line through the tissue.

2. The apparatus for non-invasive atrial septal defect repair according to claim 1 wherein the positioning member is an expanding member and is configured to expand when positioned within the Atrial Septal Defect.

3. The apparatus for non-invasive atrial septal defect repair according to claim 2 wherein the expanding positioning member is an inflatable member.

4. The apparatus for non-invasive atrial septal defect repair according to claim 3 wherein the inflatable positioning member is configured to have a smaller diameter in the deployed position at the defect than the diameter of the positioning member in the deployed position at the location that the lumen is coupled there to.

5. The apparatus for non-invasive atrial septal defect repair according to claim 1 further including at least one central lumen configured to extend through the Atrial Septal Defect, and wherein the at least one central lumen is configured to co-operate with the suture deploying lumen when the positioning member is received and deployed within the Atrial Septal Defect to secure a suture line through the tissue.

6. The apparatus for non-invasive atrial septal defect repair according to claim 5 further including a plurality of the central lumens.

7. The apparatus for non-invasive atrial septal defect repair according to claim 1 further including a plurality of suture deploying lumens suture deploying lumen coupled to the positioning member at radial spaced positions about the positioning member.

8. The apparatus for non-invasive atrial septal defect repair according to claim 7 wherein the positioning member is an inflatable and deflatable member and is configured to expand when positioned within the Atrial Septal Defect.

9. The apparatus for non-invasive atrial septal defect repair according to claim 1 wherein the suture line is configured to include an expanding suture anchor adapted to prevent the suture line from being drawn back through the tissue.

10. A suture application apparatus for tissue defect repair comprising:
an expandable positioning member configured to be received and deployed within the tissue defect,
At least one suture deploying lumen coupled to the positioning member and configured to be aligned with tissue adjacent the tissue defect when the positioning member is received and deployed within the tissue defect; and
A piercing member within the suture deploying lumen and configured to pierce the tissue adjacent the tissue defect when the positioning member is received and deployed within the tissue defect to secure a suture line through the tissue.

11. The suture application apparatus for tissue defect repair according to claim 10 wherein the positioning member is an expanding/contracting member and is configured to expand when positioned within the tissue defect.

12. The suture application apparatus for tissue defect repair according to claim 11 wherein the expanding positioning member is an inflatable/deflatable member.

13. The suture application apparatus for tissue defect repair according to claim 12 wherein the inflatable positioning member is configured to have a smaller diameter in the deployed position at the defect than the diameter of the positioning member in the deployed position at the location that the lumen is coupled there to.

14. The suture application apparatus for tissue defect repair according to claim 10 further including at least one central lumen configured to extend through the tissue defect, and wherein the at least one central lumen is configured to co-operate with the suture deploying lumen when the positioning member is received and deployed within the tissue defect to secure a suture line through the tissue.

15. The suture application apparatus for tissue defect repair according to claim 14 further including a plurality of the central lumens.

16. The suture application apparatus for tissue defect repair according to claim 10 further including a plurality of suture deploying lumens suture deploying lumen coupled to the positioning member at radial spaced positions about the positioning member.

17. The suture application apparatus for tissue defect repair according to claim 16 wherein the positioning member is an inflatable and deflatable member and is configured to expand when positioned within the tissue defect.

18. The suture application apparatus for tissue defect repair according to claim 10 wherein the suture line is configured to include an expanding suture anchor adapted to prevent the suture line from being drawn back through the tissue.

19. A method of non-invasive transcatheter atrial septal defect repair comprising the steps of:
Advancing a positioning member along a catheter into the Atrial Septal Defect, wherein at least one suture deploying lumen is coupled to the positioning member with a piercing member within the suture deploying lumen;
Deploying the positioning member within the Atrial Septal Defect to align each suture deploying lumen with tissue adjacent the Atrial Septal Defect; and
Piercing the tissue adjacent the Atrial Septal Defect with the piercing member to secure a suture line through the tissue.

20. The method of non-invasive transcatheter atrial septal defect repair according to claim 19 further including the step of advancing a repair patch along suture lines to repair the defect and securing the patch in place with the suture lines.
